(19) United States
(12) Patent Application Publication
(10) Pub. No.: US 2004/0082842 A1
(43) Pub. Date: Apr. 29, 2004

(54) System for monitoring fetal status

(76) Inventors: Vijay K. Lumba, San Jose, CA (US); Irwin Wunderman, Mountain View, CA (US); Mearl Naponic, San Diego, CA (US); John Missanelli, Lamesa, CA (US); Angela Lumba, San Jose, CA (US)

Correspondence Address: VIJAY K. LUMBA, 3077 BATES CT, SAN JOSE, CA, 95148, US

(21) Appl. No.: 10/281,041

(22) Filed: Oct. 28, 2002

(51) Int. Cl. A61B 5/00

(52) U.S. Cl. 600/338

(57) ABSTRACT

During childbirth, trauma to the infant can readily arise, ultimately resulting in fetal hypoxia, academia, and brain damage. Such unfavorable conditions can be best ascertained by real-time monitoring of the fetus' blood-oxygen level, heart rate monitoring, EKG and EEG waveforms. This invention describes a system of monitoring devices to implement such goals and maximize the potential welfare of the fetus. It furthermore allows the formation of a reference database that can correlate intrapartum events from prior births. The embodiments utilize a small-diameter sensor inserted into the birth canal through a tubular insertion rod. Through wire, fiber optics, and or using a radio frequency link, fetal monitor data can be analyzed, compared to existing data base, and or transmitted via internet. This patent details various apparatuses that allow important life-sign parameters of a fetus to be continuously monitored.

### CROSS REFERENCE TO RELATED PUBLICATIONS

U.S. Pat. No. 6,122,042 Devices and Methods for Optically Identifying Characteristics of Material Objects.

### References Cited

U.S. PATENT DOCUMENTS

| | | | |
|---|---|---|---|
| 4,149,528 | April 1979 | Morphey | 128/206 |
| 4,320,764 | March 1983 | Hon | 128/635 |
| 4,437,467 | March 1984 | Helfer et al. | 128/642 |
| 4,501,276 | February 1985 | Lombardi | 128/642 |
| 4,658,825 | April 1987 | Hochberg et al. | 128/634 |
| 5,109,849 | May 1992 | Goodman et al | 128/633 |

### BRIEF SUMMARY OF INVENTION

The present invention relates to inserting a sensor into the uterus and acquiring data to monitor a fetus prior to birth. Primarily blood oxygenation, electrical heartbeat signals, and electrical signals from the brain (if the head is accessible) can be obtained by the method(s) delineated in this patent. It is also feasible to obtain quantitative and qualitative determinations related to fetus blood, glucose, albumen, cholesterol, brain activity, video images, heartbeat and other acoustic sounds, tissue electrical and optical properties, etc. Such data can lend insight to the fetus' welfare.

The difficulty concerning such a monitoring device lies within its successful insertion and sensor adherence under adverse conditions. This invention entails mechanisms that remedy several of these problems. The advent of ASIC semiconductor chips, which perform a variety of signal processing and control functions in a minute region, make tiny monitoring devices feasible. As a result, electronic circuitry is not a primary part of the problem. One embodiment of the device described permits the continual collection of data with the same sensing system used after the infant's birth. This facilitates the ease at which data can be collected and compared.

### BACKGROUND OF THE INVENTION

Existing methods to monitor the status of trauma in a fetus typically involve data derived from electrical heartbeat or acoustic sounds recorded through the mother's abdomen. On the contrary, this patent design accesses the fetus through the birth canal using long extension tubes. In one common embodiment, an electrode formed from a helical spring coil or screw is inserted through the vagina and is screwed into the fetal body. From that electrode, voltage measurement relative to ground provides a heartbeat signal from which the clinician can obtain information concerning fetal condition. A more important parameter that can conceptually be monitored is asphyxia neonatorum. That generally requires measuring the comparative absorption of two different optical wavelengths correlating to de-oxy or oxyhemoglobin. Using a simple ratio of the two, blood-oxygen saturation can be determined. Such measurement is standard procedure after the infant's birth. Though, by that time, the infant is in an oxygen rich environment and normally does not experience significant variability in blood oxygenation with time. However, the converse is true in the case of the fetus and its oxygen needs and therefore, timing can be critical. Neurological and myocardial complications can arise from only a few minutes in a hypoxic state. Some clinical examples are hypoxic ischemic encephalopathy, meconium aspiration syndrome, acidosis, cerebral palsy, and neonatal seizures. Often the attending obstetrician has no clear indication of the onset or degree of adversity of this condition, and timing is of the essence. The problem of continuously monitoring optical variables in utero is directly linked with achieving a stable, optically acceptable sensor-attachment to the fetus. The uterine environment is quite hostile to optical sensing because of prevailing fluids, shifting due to contractions, fetal presentation, and the difficulty of early access, the variable fetal epidermis, and the presence of hemophilia, venereal diseases, or infectious biota. This invention addresses solutions to different cases posed by these problems with apparatuses consisting of an assemblage of sensors and embodiments of slightly different mechanisms that can fill the need for most circumstances.

### BRIEF DESCRIPTION OF THE DRAWINGS

FIG. 1 shows the basic method of inserting and attaching the sensor to the fetus.

FIG. 2 indicates a tissue needle attached to sensor housing.

FIG. 3 shows a hollow tissue needle with two (or more) protruding optical fibers.

FIG. 4 displays a hollow tissue needle containing a single optical fiber.

FIG. 5 shows the pointed end of a tissue needle having an internal light guide.

FIG. 6 displays a configuration with optical light guides coupled to emitters and a detector emanating in two hollow tissue needles.

FIG. 7 displays a configuration wherein the tissue needles and light guides are molded from a single metallized plastic part and then separated.

FIG. 8 shows a configuration for attaching “large area” button housing to the fetus.

FIG. 9 shows an arrangement for electronic and optical circuitry for a blood oxygen monitor that avoids penetrating the epidermis.

FIG. 10A portrays how the sensor housing of a bandage type fetal monitor is configured.

FIG. 10B illustrates a “Bandage” type fetal monitor configuration with four extended flaps that can aid attachment and provide electrical measurements.

FIG. 10C shows the bandage type fetal monitor within an insertion tool prior to being attached to the fetus.

FIG. 10D indicates details of the insertion tool for the bandage type fetal monitor.

FIG. 11 indicates typical electronic and optical circuitry that could comprise the components for, (or an ASIC chip of) a fetal monitor and computational system.

### DETAIL DESCRIPTION OF THE INVENTION

Numerous different devices comprising this system are required because all childbirth may be different. It is therefore important that any attending gynecologist have a matrix of devices yielding slightly different functions as deemed necessary at the time of delivery. An important ancillary part of this patent and system relates to data collection and the build-up of a world wide database. Accordingly, each of the similar but slightly-altered-function-devices presented herein would be respectively encoded electronically (as to determine which device it is), filling one or more fields of the database. The entire system of these devices (with subsequent others added in the future), in connection with the receiving electronics, algorithms and optional Internet interacting computer, are all considered part of the patent. In that manner the gynecologist would be granted available norm values for typical parameters to inter-compare with the specific case at hand. That also yields the significant benefit that many parameters can be measured without knowing precisely what those variables signify, so long as they are acquired via the same means (i.e., by the same devices and circumstances). Such parameters can be something that the computer can later determine by statistical analysis. The computer can “flag” seemingly relevant differences in electrical, optical or whatever measurements it “data mines” from the ensemble of prior births previously acquired through a similar system. If or when such differences manifest, the computer can warn the attending clinician that can make judgments as to appropriate restorative action to be taken. This invention is thus meant to cover an entire system for fetal monitoring, storing, and analyzing data. Part of the objective is to add data to and compare it with the database, plus making expert system type precautionary recommendations where feasible and possible ameliorating responses. Each sensor embodiment provides a slightly different “probe for the system” and will usually require its own calibration and internal identification for recognition by the system.

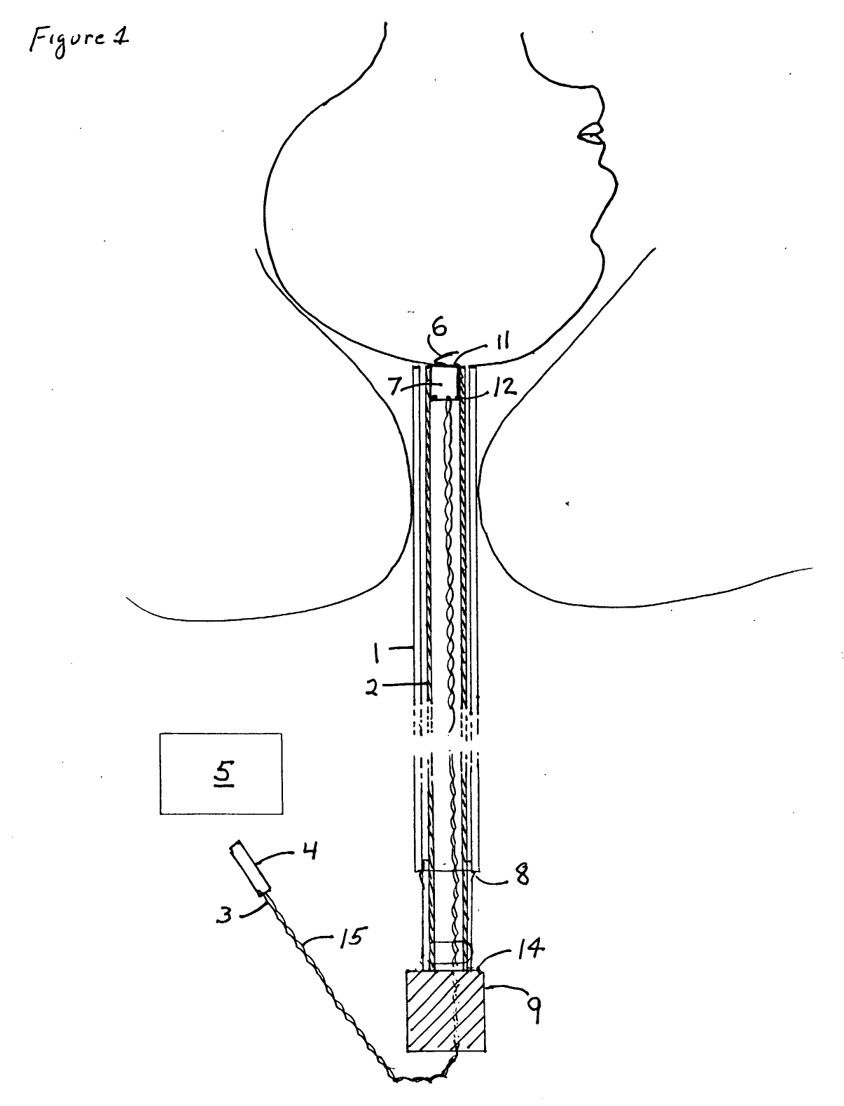

FIG. 1 shows a typical method of EKG type electrode installation that is currently in use and the configuration characterizes at least one embodiment of this invention. An electrode is attached to the fetus at the end of an extended and bendable guide-tube (1) and drive-tube (2). The process allows the removal of the guide-tube and drive-tube insertion mechanism after the fetal-monitor housing is attached to the fetus. The signal leads (3) with terminating connector (4) extend from the monitor through the center of drive-tube (2) as the drive-tube and guide-tube slide away over the signal leads (3). The terminating connector (4) can then plug into the receiving electronics (5) to complete the system hookup. The receiving electronics will generally include a computer for data processing, storage, and analysis. That computer can optionally be connected via the Internet to a “central database computer” that contains compiled and interactively available data from previous births, from all over the world. The database can provide “norm conditions” about measured parameters in juxtaposition to those of the present birth.

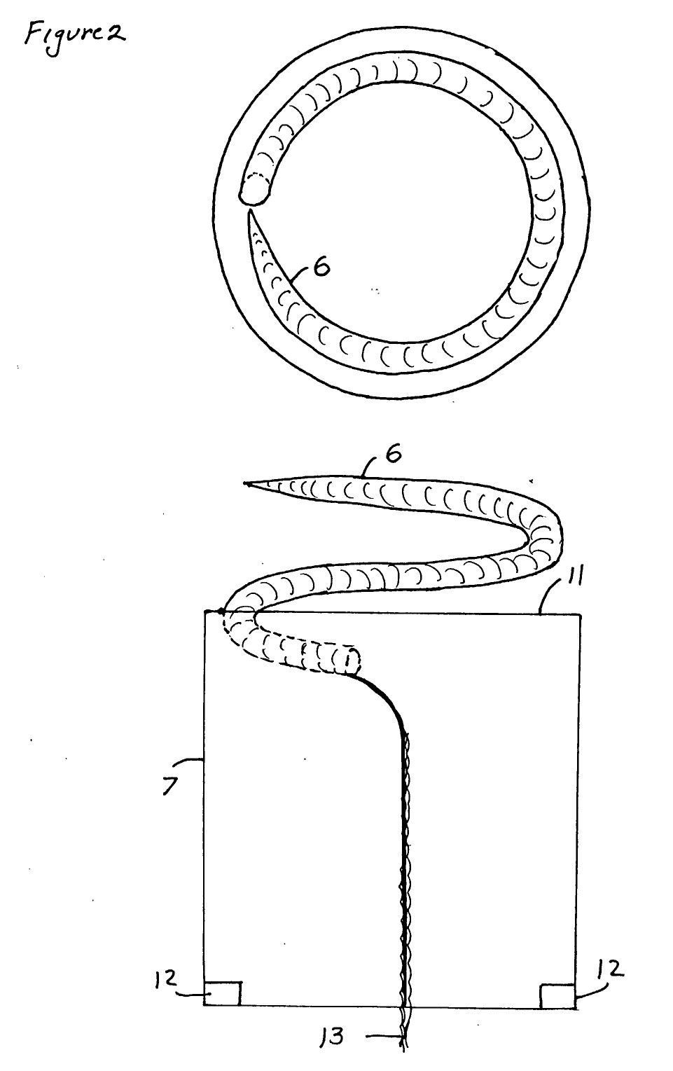

The currently used method for securing a helical electrode tip to the fetus usually employs a single pointed “helical screw” also called a tissue-needle in this patent (6) shown in detail in FIG. 2. It protrudes from the monitor support structure also called the housing or sensor (7) and literally screws into the fetus by rotation under slight pressure. The sensing housing is nominally about 5 to 6 mm in diameter and 8 to 10 mm long. At the time of vaginal insertion, the drive-tube has its helical-screw electrode retracted from the distal end of the guide-tube, as limited by the insertion stop (8) at the proximal end of the drive-tube. After the guide-tube presses against the presenting fetal part, the drive-tube is advanced forward past the stop (8) to sense contact with the fetus. The drive tube, through its drive handle, (9) is then rotated one turn under controlled pressure. The sensor housing, (7) with tissue-needle electrode in the shape of a helical spring screw (6) protruding from its front contact surface, (11) penetrates the epidermis of the fetus at the distal end. The drive-tube engages the sensor through interlocking notches in each (12). Drive and guide tubes being about 30 centimeters long are then slid off the protruding wire (13) and connector (4), and discarded. Maximum engagement of the drive-tube into the guide-tube is limited by the final stop (14). Electrical contact with the fetus is maintained through the signal wires with reference electrode (15) serving as a shield. While this method is satisfactory for electrode attachment, it has the disadvantages in that only one point of attachment occurs at the proximal end of the helical screw electrode. If the sensor housing (7) is disturbed or jarred, the front contact surface (11) of the housing may not remain flush with the fetus epidermis. This can be further aggravated if the point of epidermis penetration has significant wrinkles, hair, etc. Large bending and rotating stresses can occur at the single point of tissue penetration thus producing greater wound damage internally than at the epidermis or the electrode diameter itself. It is difficult for a clinician to establish when the helical screw is fully engaged leading to excess twisting that churns the fetal tissue and weakens the bond.

The electrode configuration does not provide any optical spectrographic information about the fetus. Such added information could be extremely useful in terms of providing levels of blood oxygen, general variances from norms, glucose, albumen, cholesterol, and a visual picture of the contact region, etc. Fetal pulse oxygenation can be one of the most useful parameters to monitor at early stages of the labor/birth process. The configuration only measures EKG type signals. It does not supply needed blood oxygen information or EEG signals. At this stage of pre-birth, fetal blood oxygenation and these other parameters can be important indicators of trauma. Only a single electrode contacts the fetus so that measurements are only possible with respect to ground (the mother). Signals from her heartbeat add ambiguity. Two or more of contacting electrodes would allow differential measurements of local electrical (or brain activity when attached to the head), in addition to measurements relative to ground. Multiple electrodes would permit a greater variety of additional electrical tissue measurements useful to indicate fetus status. These include comparison of norms of real/imaginary components of tissue impedance vs. frequency; rise/decay time analysis of voltage and current from a step applied current or voltage, response to various electrical stimulation, etc. Such measurements are meant to be inclusive under this patent whenever active signals are applied to the electrodes with parameters being measured, in addition to passively extracted signals from the tissue. More than one electrode permits sequential multiplexing of the electrodes to perform those various other functions and they are implied herein when multiplexers are utilized.

If an electrode is used to pierce the epidermis, it might as well generate more information about the other side of the skin. The epidermis introduces considerable noise and variability to electrical and optical signals. Tissue and cell measurements that go through it generally encounter a much greater degree of uncertainty and ambiguity than those that avoid penetrating the skin. Birth fluids on the outside of the epidermis also obscure determination of tissue properties inside the skin.

As required for early attachment to the fetus, extended visibility into the birth canal can be poor. To facilitate adequate illumination and clearly establish the status of the presented fetus part would generally require greater opening than the guide-tube diameter. Thus, because of poor illumination, insertion of a fetal monitor unit might be postponed until a later state of labor. The degree to which the front contact surface of the monitor makes good but not excessive contact with the fetal epidermis at a convenient location is often a difficult parameter to control. Under this patent external illumination can be employed to enhance controllability of these contact issues. Availability of illumination at the distal sensor end makes feasible a miniature CCD, CID or CMOS type camera and lens within the sensor housing (7). Because consecutive image frames can be taken with a different externally applied wavelength light using either the guide tube (1) or drive tube (2) as light pipes, hyperspectral imaging of the attendant region becomes possible. The clinician can then have improved choices about the point of fetal contact, prior to actual contact. Judicious determination can be made as to whether contact should be made then and there. A coherent bundle fiber optic provides a further method for obtaining images with the camera being at the proximal end of the fiber optic.

A full turn is required to engage the single helical screw while a clinician's human hand can only rotate about a half-turn in a single operation. This either requires two consecutive hand twists, or twisting the drive handle (9) between thumb and forefinger. Both these methods provide less of a vernier on pressure sensing and the feel of precisely full engagement than for a half-turn of the hand under one single continuous hand-twist. Requiring more than one wrist twist one looses calibration of exactly how much rotation was applied and the tendency is to overcompensate and mash fetal tissue.

The exact depth of penetration of the electrode tip (6) inside fetus tissue can experience variability due to requiring one full turn of advance before the front the contact surface of the housing (11) contacts the epidermis. Excess turning can churn and squeeze the tissue adding unnecessary trauma. Were a deficient amount of turns utilized then required, bond to the fetus can be poor. In general, the penetrating end-depth of the tip becomes less variable by approximately the square of the number of turns required. The ability of a camera at the front contact surface to provide an image of the contact region can greatly diminish these and other pragmatic difficulties toward adherence of the sensor. Even the retro-reflected signal magnitude from illumination through one guide tube and return from the other can be of help to appropriately contact surface (11) with the fetus.

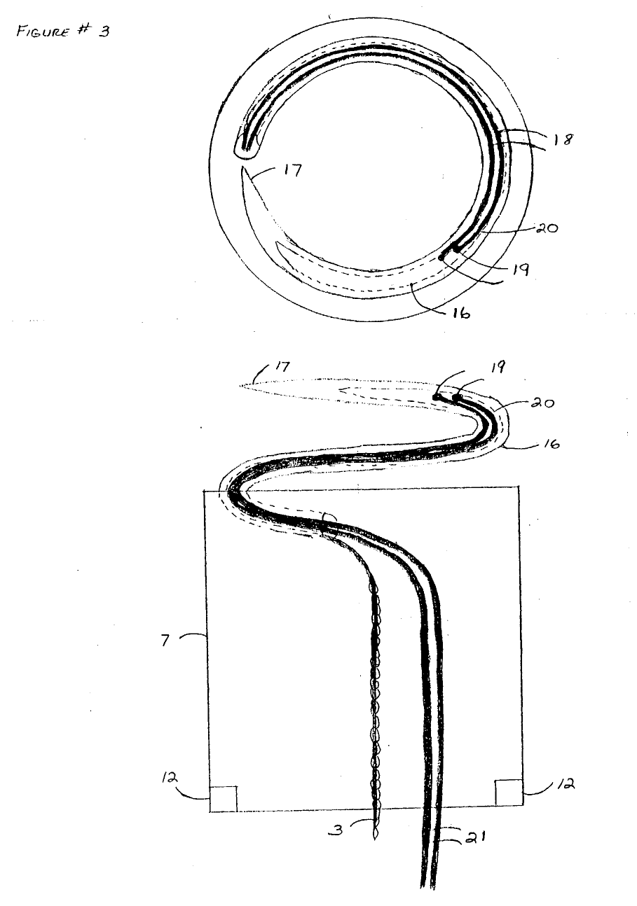

New and novel embodiments for affixing the sensing monitor housing to fetal tissue are included under this patent, as well ability to monitor other useful data than electrical heartbeat. The first embodiment of this patent to be discussed is portrayed in FIG. 3. In it, the helical screw electrode 9tissue needle) of FIG. 2 can be thought of as formed from hollow tubing (16) brought to a pointed tip (17), but otherwise similar to existing utilized helical electrodes. Two flexible optical fibers (18) advance axially within the tube's hollow region. They emerge from the hollow tube electrode toward the tip end through one or two holes (19) in the tubing wall. The fibers can be epoxied in place (20) with the ends emerging from the helical electrode polished off at the distal surface where they protrude through the hole in the cylindrical wall (19). The cylindrical tube electrode (tissue needle) electrically connects to one of the signal leads (3) that joins the housing (7) to the terminating connector (4). Extending from the proximal end of the housing (7) are the two optical fibers (21) from within the electrode, which go to a small-diameter optical connector similar to, or integrated with, the electrical connector (4). After inserting the electrode into the epidermis, the guide-tube and drive-tube are removed from the wires and both connectors insert into the receiver electronics circuitry (5). One fiber is energized by two or more different-wavelength emitting LED's or laser diodes and the output from the other fiber impinges on an optical detector, often a silicon PIN detector. The LED's or laser diodes can alternatively be Vexels, OLEDS, quantum dots or polymer type LED's, etc. The letters LED herein signifies any of those polymer photon emitters or junction luminescence type alternatives. The peak wavelengths are judiciously selected for functions to be performed. The LED's are actuated sequentially, individually or in combination, each being modulated on and off at a relatively high repetition rate during “its on-time”. For blood oxygen measurements, the different emission wavelengths of the LED's or laser diodes are appropriately selected for maximum discrimination to evaluate hemoglobin oxygenation by ratio of their optical transmission through the tissue. Typical wavelengths are 730 nm and 890 nm. The sequential radiation emanating from the distal end of the emission fiber scatters through the tissue and bears some signature of the tissue and its constituency. Some fraction of that radiation ends up collected by the other optical fiber thereby reaching the detector in the receiver electronics (5) at the proximal end. The detected signal is amplified, (typically via a trans-impedance amplifier) and synchronously detected in cadence with each LED or laser's on-off modulation. The ratio of the two or more different wavelength signals can thereby bear a relationship to blood oxygenation in the fetus. Thus, with similar exterior geometry, procedure and expertise level to employing existing single helix fetal-electrodes, the enhanced device described with a light pipe in the helical screw can also provide blood oxygenation information. This allows the electrode to also furnish a totally different function than electron conduction and represents one new and novel realization of this invention.

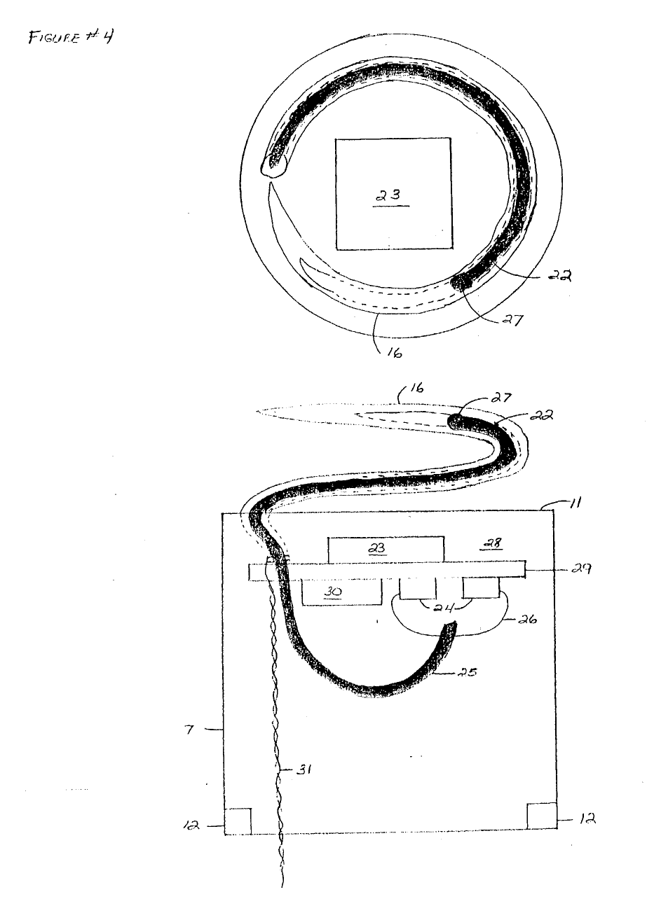

Another embodiment of this invention (FIG. 4) utilizes only one larger-diameter-fiber (22) within and exiting the hollow metal electrode. The combination is also considered a tissue-needle, which for purposes of this patent can be a pointed elongated shape comprised of metal, or of clear dielectric type material like plastic or glass, or of combinations of metal and clear material. Generally, the clear material is either primarily interior or primarily exterior to the metal. Tissue-needle functions can include one or all of the following: to penetrate tissue; to serve as a light guide, to protect an interior light guide, as an electrode, as an appendage to hold the sensor onto an object, to establish fetal contact by electric or optical means. A specific embodiment may utilize any feasible combination of these functions and this potential multiple-purpose-functionality is considered a novel part of this patent. For the embodiment of FIG. 4, the increased fiber diameter for the same external electrode diameter allows greater optical transport of the emission sources. The additional optical throughput permits the configuration to readily function with LED's, Vexels, OLEDs, Quantum dots, or polymer type LED's, etc., as alternative to laser diodes. The additional signal-strength also makes feasible various emitter/detector configurations that circumvent the need for optical fibers to connect to the proximal electronic receiver (5) via fibers (21). In these descriptions the phrases optical fiber, fiber optic, light guide, light pipe are used more or less interchangeably, although the former two generally have a lower refractive index exterior cladding. Light pipes and light guides can have either an air or reflective metal exterior surface or a lower index cladding. For short distances between elements in the sensor and the fetal tissue, the differences can be insignificant. Fibers with cladding would typically be employed to bring optical signals greater distances through the birth canal. Here, a large area photo-detector (23) viewing a large solid angle is embedded within the housing (7). The relatively large sensing area can minimize the effect of infant hair and other epidermal anomalies. Although the system gathers optical information, only electrical wires need run from the housing to the proximal receiver electronics circuitry (5). One or more emitting (24) crystalline LED's, vexels, polymer LED's, or lasers couple into the single fiber (25) through a clear dielectric coupling medium (26). (Multiple LED's would be used for blood oxygen and other determinations.) A fraction of the emitted photons reach the electrode end of the fiber at the single hole (27) in the tubular electrode (16). That optical aperture may be either at the end or along the side of the tissue-needle coil. That optical port faces the front contact surface (11) of the housing (7). The emitted photons scatter through the tissue and the relatively large area optoelectronic detector (23) embedded in clear encapsulant (28) within the housing detects some fraction of them. The detector creates an electrical signal proportional to the rate of impinging photons. The electronic components are mounted on a PCB {Printed Circuit Board (29)}. The detector amplifier (30) brings a replica of the transmitted optical signal to a relatively high electrical level before being returned, along with additional wires (31) to the electronic receiver for the electrode, power, and for driving the LED's plus other signal wires (15). Discussion here generalizes certain detailed distinctions between all the possible embodiments brought out in the claims. It is also feasible to bring the single optic fiber within the electrode all the way to the electronic receiver circuitry so that the LED's or laser diodes are located at the receiver instead of within the sensor housing. In this case, it is possible to employ a scanning spectrometer so the optical emission comprises a scanned range of wavelengths across a continuous spectrum. This can permit monitoring other tissue constituents along with blood oxygenation in the fetus. As indicated in U. S. Pat. No. 6,122,042, more sophisticated analysis of fetal tissue can be performed via an array of different-wavelength LED emitters, instead of or alternative to a scanning spectrometer. As a result, density-of-state comparisons of tissue-cell molecules can be performed through IDEA probe type spectroscopy as described in that patent but also an applicable technique implied herein. Although extra circuitry would be required within the housing (7), or receiver electronics (5), the information derived can be very useful. Many different wavelength LED's may be sequentially multiplexed individually or collectively into the configuration. Methods of epidermal penetration and attachment delineated herein enable bringing this new type spectroscopy to fetal tissue as well as to biological entities in general.

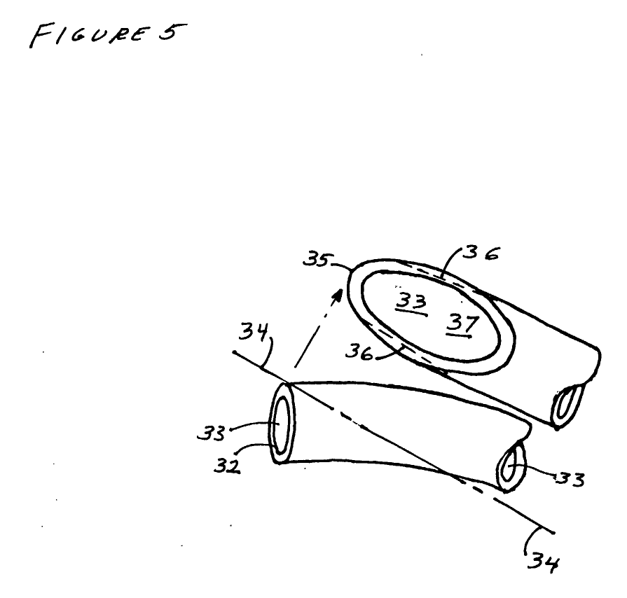

A further variation of this invention allows the electrode tip to be fabricated in a different manner than shown in FIGS. 3 and 4. See FIG. 5. Here, the ID (32) and OD of the metal electrode tubing is everywhere maintained constant with optically clear light-pipe material or optical fiber filling the interior of the tubing (33). After formation of the electrode in the shape of a helical coil, that coiled tubing with optical material inside is cut at a steep oblique angle (34) to form a sharp point (35). The side edges of that cut are “rounded” (36) so that only the tip remains sharp. Optical radiation would emanate from the elliptical face of the cut region of tubing where the optical surface is exposed (37). That optical surface faces the detector located within the front contact surface (11 ) of the housing (7). The optical light pipe could be either clear dielectric material within the reflective tubing walls, or clad optical fiber glued within the tubing.

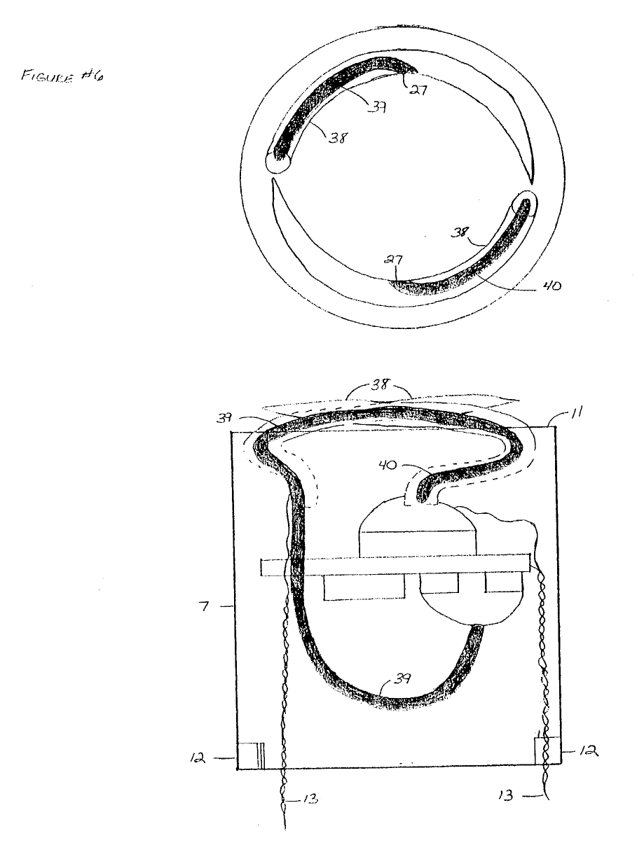

A further embodiment utilizes two symmetrical, (38) rather than a single helical-screw electrode, each of which has a light pipe within it. See FIG. 6. This would require only a half-turn twist of the drive-tube, rather than a full turn, allowing enhanced human-hand “feel” of increased pressure resistance from electrodes penetrating the tissue. Twisting and advance that occurs at the front contact surface (11) of the housing can be more readily felt and judged. The sensor housing (7) would also sustain more rigid adherence to the epidermal surface when held in place from two diametrically opposite positions. One emission fiber (39) brings two or more optically emitted wavelengths to that electrode's optical output as occurred in FIG. 4. If emitters are not included in the housing, the proximal end of the fibers could alternatively return to the electronic receiver circuitry along with the other lead wires (13). The second detector fiber within the other electrode (40) brings the transmitted signal back to the detector either embedded within the housing as shown in FIG. 6, or at the receiving electronics. Both holes in the pair of electrodes (27) face each other so that transmission is primarily through tissue without any intervening epidermal layer. That enables the most accurate and stable determination of blood oxygenation because only homogeneous blood-laden tissue is in between emitter and detector light pipes. A further advantage of this configuration is that much, or all, the electronic circuitry can be included within the housing. Such miniaturized circuitry typically consists of: drivers for the LED's, detector amplifier, synchronous detectors, ratio circuitry, analogue to digital converters, timing control, and if desired a radio frequency (RF) emission link to the remote receiver electronics with computer and monitor when a battery is included. Close proximity of all the circuitry within the housing can reduce Electromagnetic Interference Noise, which is often quite variable in hospital environments. The entire circuitry, less LED's, can be a single ASIC chip, or separate semiconductor chips for specific interconnected functions. Since readings need only be taken many seconds or minutes apart, a very minute battery providing nominally 1.5 to 3 volts could adequately power the unit for up to 1000 readings or 20 hours. This would allow each optical reading to constitute an average of perhaps 2000 over-samplings of 20 microsecond on-times. A wide range of other on-times and oversamplings are feasible as warranted. The weight of the entire housing attached to the fetus need not exceed 0.5 gram. The unit may be turned-on either by the applied torque of the drive-tube activating a minute switch (12) on the housing, or by electronically sensing the inter-electrode impedance.

It is noteworthy that an optional use for the guide-tube and drive-tube can couple illumination from the uterus exterior to the region of presentation on the fetus and back to the clinician. A bright light source matched to the proximal annulus of the guide-tube could provide illumination at the distal region of that tube near the fetus. The degree of returned illumination coming back along the drive tube would be affected by how close the front contact surface (11) of the housing (7) is to the fetal epidermis. This would be particularly true if one small perimeter region of the housing acted as a front-to-rear light guide by being clear dielectric material. Then, the returned illumination along the drive-tube would increase markedly when the front surface of the housing approached the epidermis. By having a small viewing area at the proximal end of the drive-tube, the obstetrician/gynecologist would receive an optical signal indicative of the proximity of contact between the front contact surface and the fetus. This grants another useful parameter for sensing contact, one of the more tricky operations associated with this type of fetal monitoring. A further embodiment is the inclusion of a minute CCD camera within the housing that makes use of the externally supplied illumination to provide a display of the contact region.

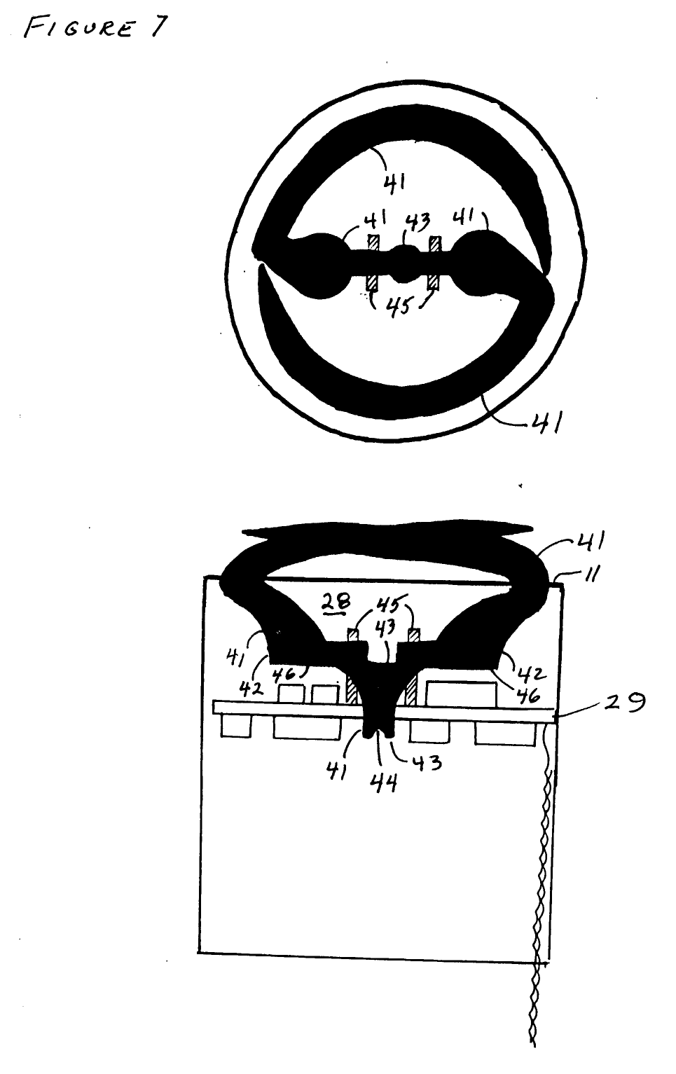

While the electrodes with internal light pipes have been discussed as fabricated from tubing with optical fibers inside, they can also be fabricated from clear plastic, metalized for electrical conduction on the outside. See FIG. 7. This adaptation can be used not only for fetal monitoring, but also for electrical and optical evaluation of all type objects, particularly those readably penetrable by needles, and such applications are intended for inclusion under this patent. Strong flexible plastics like Lexan can be injection-molded (41) into an optimized helical screw or other configuration that widens where embedded (42) below the front of the housing. Each such light pipe electrode could be molded separately, or molded joined together by an attachment bar (43) that gets drilled (44) or sawed out after final encapsulation, with the hole then filled by an opaque material to prevent cross-talk. This permits highly accurate and rigid registration of both electrodes and very precise and repeatable optical properties for the light pipes and electrodes. In the figure, the molding is shown in dark black with a cylindrical rod at the center (44) connecting both electrodes. The rod fits precisely through a central hole within the PCB (29) during assembly. After objects above the PCB are encapsulated with clear dielectric material (28), the cylinder is drilled out (44) from below, electrically separating the two electrodes. Opaque epoxy then fills the drill hole keeping both halves optically and electrically separate. Metallization around each electrode below the front contact surface (11), and an opaque mid-housing optical barrier prevent unwanted cross-talk from emitters to detector. The optical barrier is not shown to prevent crowding the drawing. Any opaque-partition configuration that separates the clear dielectric into an emitter compartment and a detector compartment would suffice. The plastic-based electrodes have the advantage of less weight, of having optimum optical light pipe shape for maximum throughput in the region of tissue desired to be instrumented, lower thermal conductivity incurring less fetus agitation, and of furnishing excellent physical precision and registration. It is relatively easy to achieve any desired value of diameter, axial direction, cross section, spring tension, optical window region, etc. along the axis of the helical screws. If desired, barbs to prevent dislodgment can be included. Such electrodes could be “spring loaded” within the guide-tube for example, so that they expand radially outward to a greater diameter helix when extended distal of the guide-tube. The tissue-needle points could also be disposed to spring out both radially and forward of the front contact surface thereby protruding into the tissue to circumvent necessity to screw into tissue. Such “spring loading” could also prevent re-use of each sensor and enable enhanced grip onto the fetus. By masking the metallization process, the non-metalized fetal regions of light pipe along the tissue-needle face each other and may extend as far as desired along each helical turn, when helical shaped needles are employed. Crimping perforated contact connectors (45) extending from the PCB to the electrode metallization can readily achieve contact with the electronic circuitry. When squeezed by a crimping tool, or when the plastic is pressed into place, they could easily penetrate into the metallization and plastic, as compared to messy soldering to helical-screw wires or tubing. Non-metalized optical faces of the light pipes below the front face (46) can be large and readily positioned conducive to constraints imposed on LED and detector locations confined on a PCB (29) circuit board.

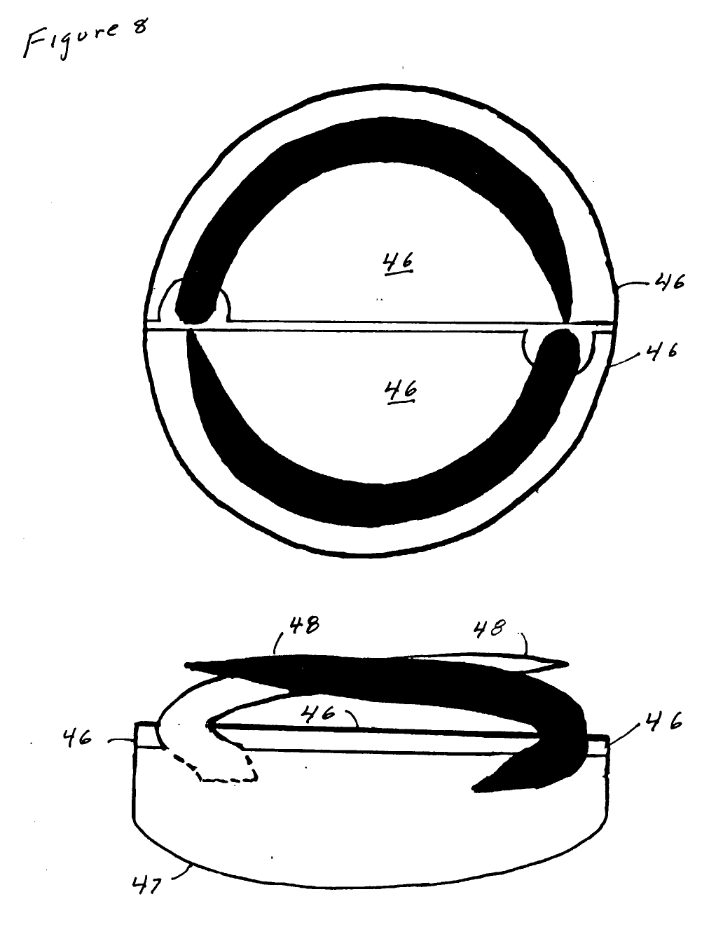

A further embodiment of this fetal monitoring system utilizes a transparent medical adhesive on the front surface of the housing to enhance adhesion to the epidermis. This grants a design where the diameter of the housing can be doubled or tripled for example, while for the same volume, its protrusion from the epidermis might be ¼ to 1/9 as great. This then allows the overall housing shape to become a tapered mound as shown in FIG. 8, and less likely to be physically bumped or dislodged. Here medical adhesive (46) helps to bond the larger area “button housing” (47) onto the fetus. This can be advantageous in traumatic childbirth where likelihood of impact increases and breaking open a region of the infant's tissue could be a source of injury and infection. The electrodes (48) can be with or without internal light guides. With sufficient bonding from adhesive, the electrodes shown curved with or without internal light pipes might also be one or more straight pins (not shown) protruding only a few millimeters normal to the front contact surface (11). Slight barbs, extractable by later stretching the skin after birth, can provide sufficient holding strength in conjunction with the adhesive. The barbs can provide one or more electrode contacts, and twisting the helical screw electrodes would be unnecessary, materially reducing the danger of surface wounds, mashed tissue and a poor bond. With either curved or straight electrodes that are metalized plastic as shown in FIG. 7, the entire range of blood oxygenation, heartbeat, EEG, tissue impedance properties, etc, can be determined.

In certain circumstances, the danger of epidermis puncture or wound damage can be so great the obstetrician/gynecologist may choose to only monitor blood oxygenation and forgo the tissue-penetrating electrodes and electrical measurements. This could be particularly important when there is concern for hemophilia or infectious biota like HIV, syphilis, gonorrhea, genital herpes, hepatitis B, or hemolytic strep. Than the preferred adherence method would be exclusively through bonding adhesive and the potential 4 to 9-fold greater contact area becomes quite beneficial. No epidermal penetration of the fetus is required and the small button attachment is less likely to get hit or dislodged.

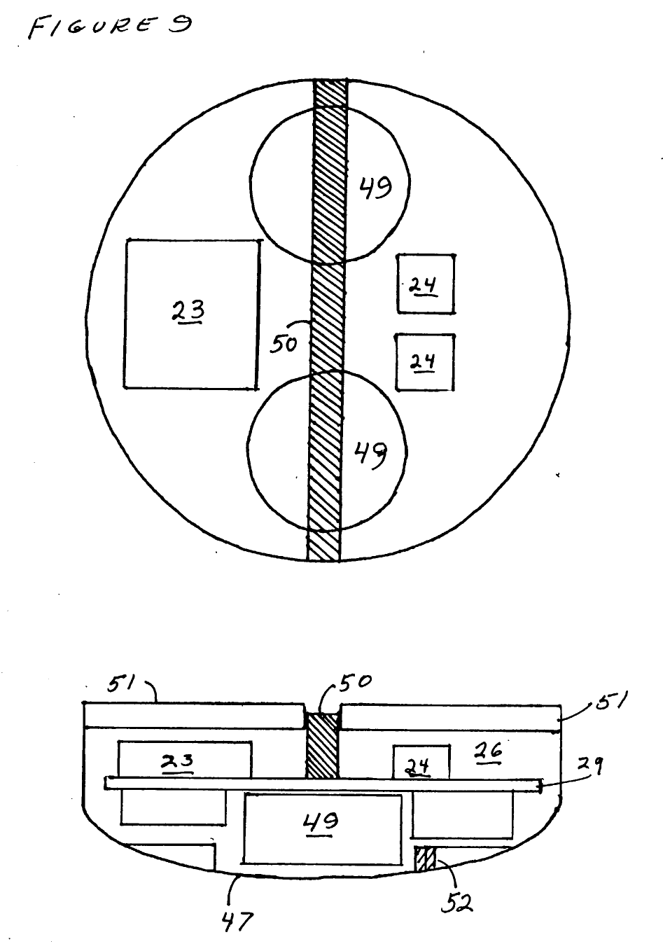

FIG. 9 shows an embodiment for a fetal blood oxygen monitor that avoids penetrating the epidermis. That arrangement uses batteries (49) and RF coupling to an external receiver, (though it is obviously also feasible to bring out flexible wires or light pipes for that purpose). That isolation, devoid of wires or optical fibers, plus the small protrusion yields a fetal monitor system with minimal drawbacks, once appropriately inserted. The detector (23) and LED's (24) are mounted on a PCB (29) in a region of optically clear encapsulation (28). An opaque optical barrier (50) straddling the full length of the housing prevents cross-talk from emitters to detector within the housing. Clear adhesive (51) on either side of the barrier provide the surface bond while allowing light penetration. Synchronous optical-radiation signals reaching the detector must arrive by scattering through fetal tissue and the adhesive area above the detector. The absorption ratio of the two detected wavelengths can indicate blood oxygenation of that tissue. The insertion method is the same as depicted in FIG. 1, but the drive and guide-tubes are either of greater diameters, or have increased diameter at the distal end. Notches and the insertion turn-on switch (52) are merely topologically altered to accommodate the different form factor of the housing (47).

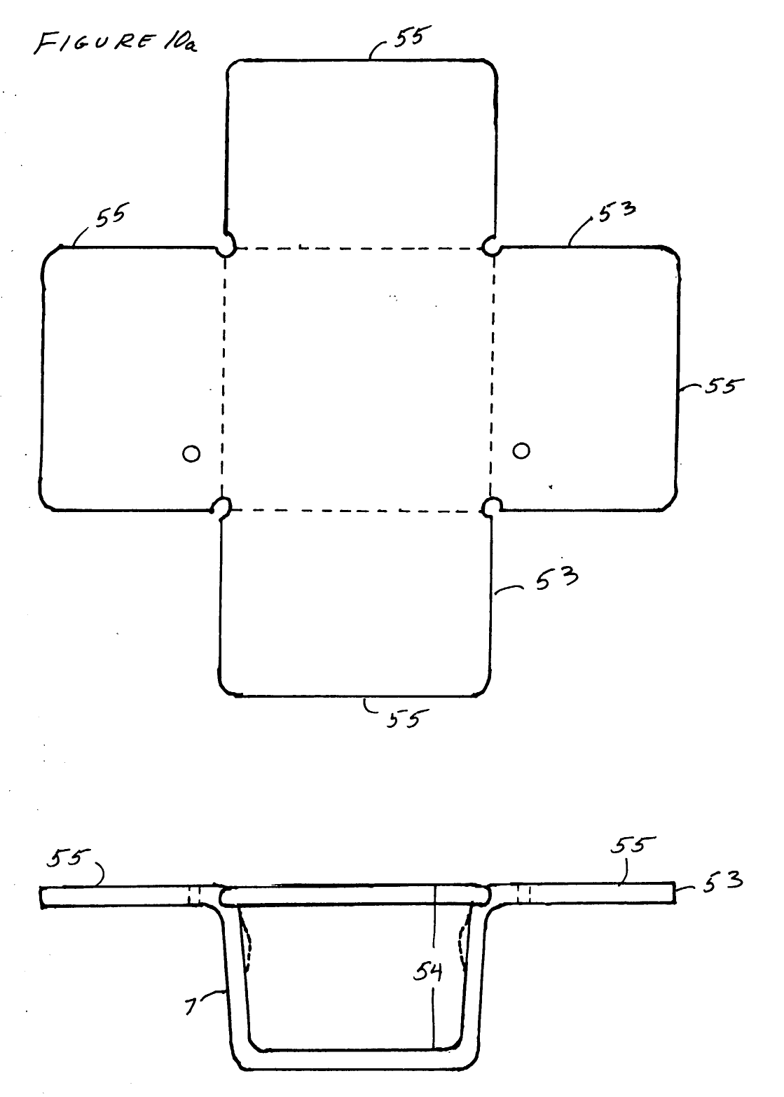

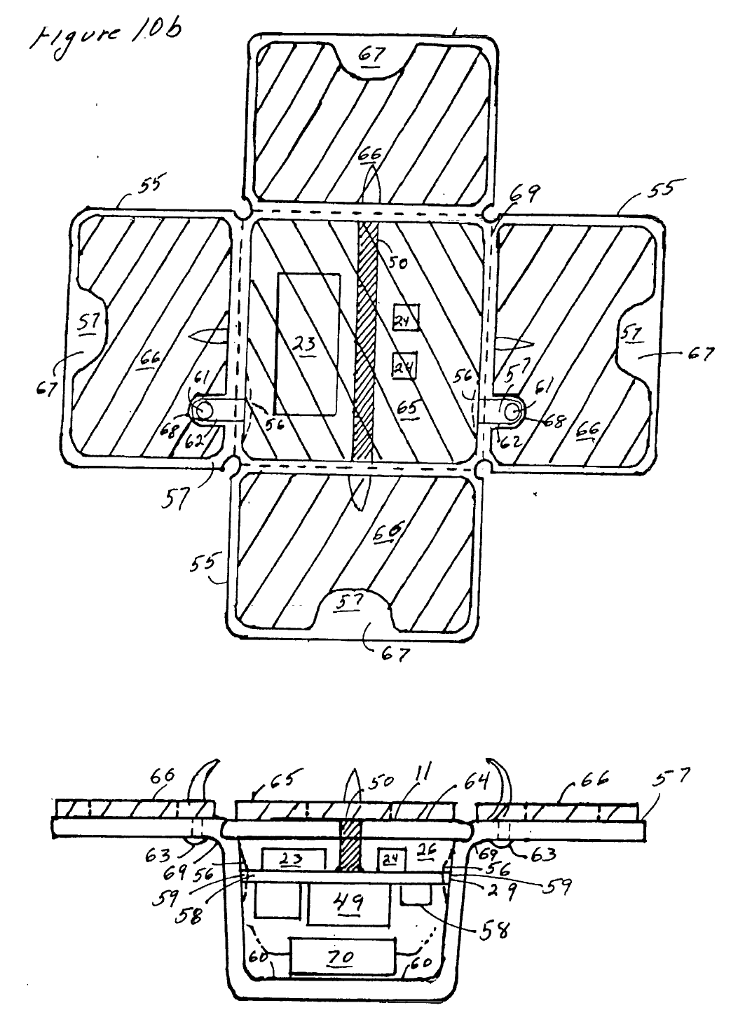

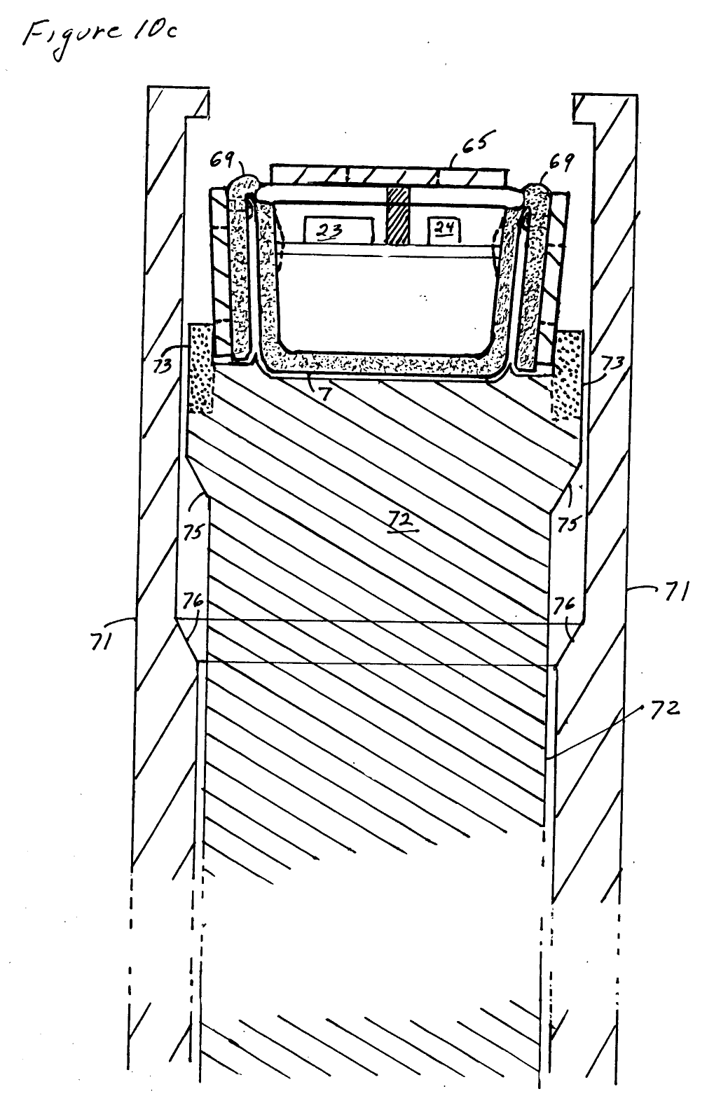

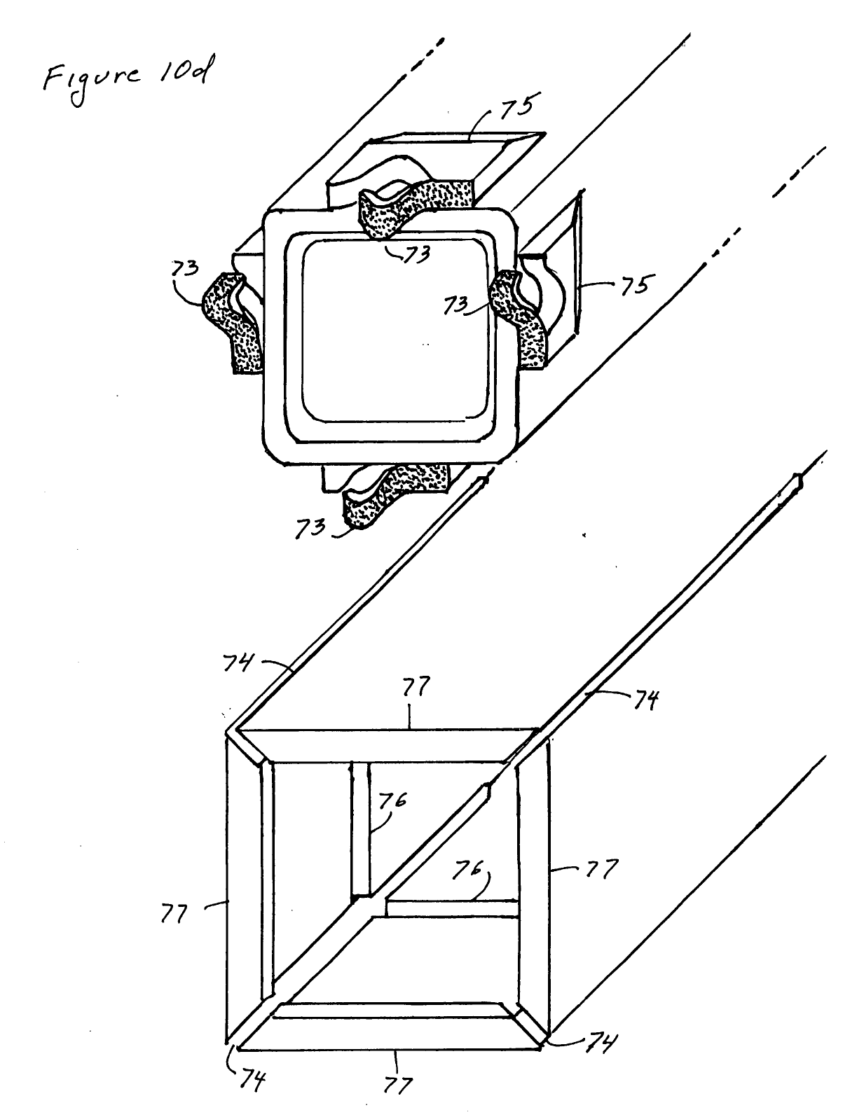

Still another embodiment that does not pierce the epidermis utilizes RF coupling and allows more of a button-sample type sensor. It can monitor blood oxygenation and provide EEG type signals. Moreover, after birth, attachable power supply leads can augment the batteries so that the infant may be computer monitored without interruption for days, weeks, or months. This incarnation, called the bandage, is configurable in various ways. An exemplary assembly arrangement is described below. FIG. 10a shows a die-cut or laser-cut pattern (53) of thin opaque sheet plastic that will be thermoformed to become the exterior housing (7). The plastic is nominally ½ mm thick and similar to continuous-plastic hinge-material. The central square region sustains a deep drawn cavity depression of depth about 6 to 9 mm (54), as illustrated in the lower part of FIG. 10a. FIG. 10b provides further illustration. The selected plastic material is such that each of the four exterior “flaps” (55) can hinge (swing) back down roughly parallel to the vertical side of the housing. When released, its retention returns it to the original shape of FIG. 10a with flaps extended. Two slight Gaussian-curve shaped depressions (56) are asymmetrically located on opposite sides of the housing to be later utilized for power supply contacts that grasp into those depressions. When the configuration is to employ electrodes, the entire topside of the housing is metalized (57) through an appropriate mask by vacuum deposition or sputtering. Electronic and optical circuitry similar to that of FIG. 9 would then be inserted into the housing cavity as a sub-assembly (58), as depicted in the lower part of FIG. 10b. Each metalized flap can become the base of an electrode. The masking would maintain electrical separation between those electrodes while bringing a conductive strip from each flap's electrical contact region into the deep-drawn area (not shown) to where the PCB edges will touch (59) along vertical sides of the housing respectively. Metallization of the inside bottom section is masked (60) to form a RF antenna that also makes contact to the PCB edge. Metallization from around both holes (61) in the two flaps is masked to isolate those leads for separate contact to the PCB edge (62). They can connect to an external power supply through a contact (63) at the top of those holes.

The volume below the PCB can be encapsulated with clear or opaque material, but exclusively clear dielectric material (26) is used on the LED/detector side. That front contact surface of the sensor (11) is encapsulated with clear material (64) to a surface flush with the metallization on the extended flaps, and clear medical adhesive (65) covers that clear surface. The flaps are covered with similar thickness conductive medical adhesive (66) that need not be clear. A central exterior region of adhesive on each flap is deleted (67), as well as regions surrounding the external-contact power supply holes (68). The hinges (69) allow the flaps to flex, but their normal position is extended radially outward. A normally closed reed relay (70) encapsulated flush within the bottom of the housing is in series with the batteries (49). With no magnet just below it, the unit will be on and transmitting multiplexed RF encoded to characterize the differential voltages between electrodes (or other desired electrical measurement functions), plus optical throughput from both wavelength emitters to the detector. When attached to the fetus those signals provide electrical brainwave signals plus pulse oxymetry information, from which heartbeat can also be determined. A fetus has not formed a rigid optically opaque cranium so with little extra complexity additional LED wavelengths responsive to brain tissue can be utilized besides wavelengths optimized for blood oxygen. Such a monitor can furnish continuous information from pre-natal through the first months of life. If the internal batteries are rechargeable, the infant can be intermittently electrically disconnected for 10 to 20 hour periods without loosing continuous information. Depressions (56) enable the contacting clip of an external power supply to adhere to the housing while supplying power and recharging the batteries. Adding to these various presented possibilities is the ability to include a miniature acoustical pickup at the housing to provide acoustical-through-ultrasonic signals. This provides another source of heartbeat information and can also pick up ultrasound being clinically applied to the mother's abdomen. Contemporary nanotechnology microphone-type devices could readily be included in the sensor.

FIG. 10c shows an embodiment of this patent rendering the housing (7) and insertion apparatus comprised of a guide tube (71) and drive rod (72) before adhesion to the fetus. When the leading edge of the Guide tube contacts the fetal surface, the drive rod (72) can push the sensor housing (7) forward to make adhesive contact over the clear central region (65). FIG. 10d shows how the spring-like extensions on the drive rod (73) keep the flaps from swinging back to their normal outstretched state. Only after the drive rod has pressed the clear adhesive against the fetus is the guide-tube pulled back past the spring extensions to release the flaps to a flattened position against the epidermis. All flaps will swing out from the central region to contact the fetus. Forward pressure is then applied with the guide-tube moving forward to subsequently press the adhesive side of those flaps toward the fetus. All five sections of adhesive contact can thus have contact pressure exerted upon them. Optionally the guide-tube can be “widened” to the extent of the outspread flaps allowing pressure to be applied over the entire flap areas. This is achieved by having the corners of the guide tube slit open for an extended length (74). Retracting the drive rod relative to the guide-tube so that the protruding abutment edge (75) presses on the inside sides of the guide-tube (76) then deflects the four parallel sides of the guide-tube tip outward (77). Pressure is applied to the flaps at each of the extended guide-tube-end positions. The flaps can be made as long as desired since the spring extension clips can be placed anywhere on the drive rod, (or the flaps can be held back by the guide tube). The four electrode areas can be multiplexed at one interval in the control sequence to form a single connection to a signal wire for EKG heartbeat measurement with respect to ground. The multiplexing can then permit the electrodes to serve independently for brain wave signals, impedance measurement, etc. The Band-Aid thus can furnish all three types of electrical and optical monitoring (plus more) without piercing the epidermis. It is totally non-invasive.

Though the Band-Aid sensor has a relatively large attachment footprint at the presented part of the fetus, the insertion mechanism need not be much larger than for a small single-electrode contact. As shown in FIG. 10c, the flaps can be folded up (69) upon insertion into the womb. They extend outward only after the central region has adhered to the epidermis and pull-back of the guide-tube releases the spring extensions on the drive rod. Because the five hinged portions permit bending between them, the Band-Aid can stick to a contoured surface as well as, or perhaps better than to a planar surface. The flaps are not sufficiently stiff that the thickness of the adhesive on them would prevent total contact with a moderately curved fetal surface. The case of four flaps around the central housing is exemplary as the system layout. It can be a polygon with N sides and N flaps, and have a circular exterior. Though not displayed in the figures, the flaps can have any desired array of straight or curved protruding electrode or holding pins facing into the epidermis. When the flaps open and are pressed into the epidermis the pins can enable any desired degree of “grab” by pre-design/selection of their length and curvature. Thus besides the adhesive bond, a penetration bond of arbitrary degree is feasible. It turns out that, even without penetrating through the entire epidermal layer, small, very fine, slightly curved-inward protruding pins can substantially increase the bond strength. They can act almost like “Velcro” without actually being invasive through the epidermal layer. Being embedded within the adhesive with points extending outward from it anywhere from zero mm to a millimeter or two, a good bond is achievable.

Drawings and text descriptions of FIG. 10 are meant to illustrate the methodology rather than being a literal account. The insertion-ends shown in FIG. 10d for example, would likely both be intermeshing injection-molded parts into which the blunt drive-rod and guide-tube ends insert. The proximal end of the guide and drive tubes would similarly comprise an attached injection-molded “Rack and Pinion”. A lever or knob could then precisely control drive-tube position relative to the guide tube. Such a generalized installation mechanism can be used to attach Band-Aid type sensors to body parts, or for industrial inspections through an endoscope-type insertion tool. An elaboration of the Band-Aid allows flaps with multiple hinges having slightly formed depressions filled with auxiliary sensing apparatus like additional LEDs. It is noteworthy that a gynecologist having available an ensemble of the different embodiments described herein can choose an optimal configuration based specific requirements for each circumstance. Having an array of different fetal monitors as necessary for the situation can be a significant advantage. Different assemblages fit different needs for the same or very similar function and thus the array of all these embodiments constitutes a single invention.

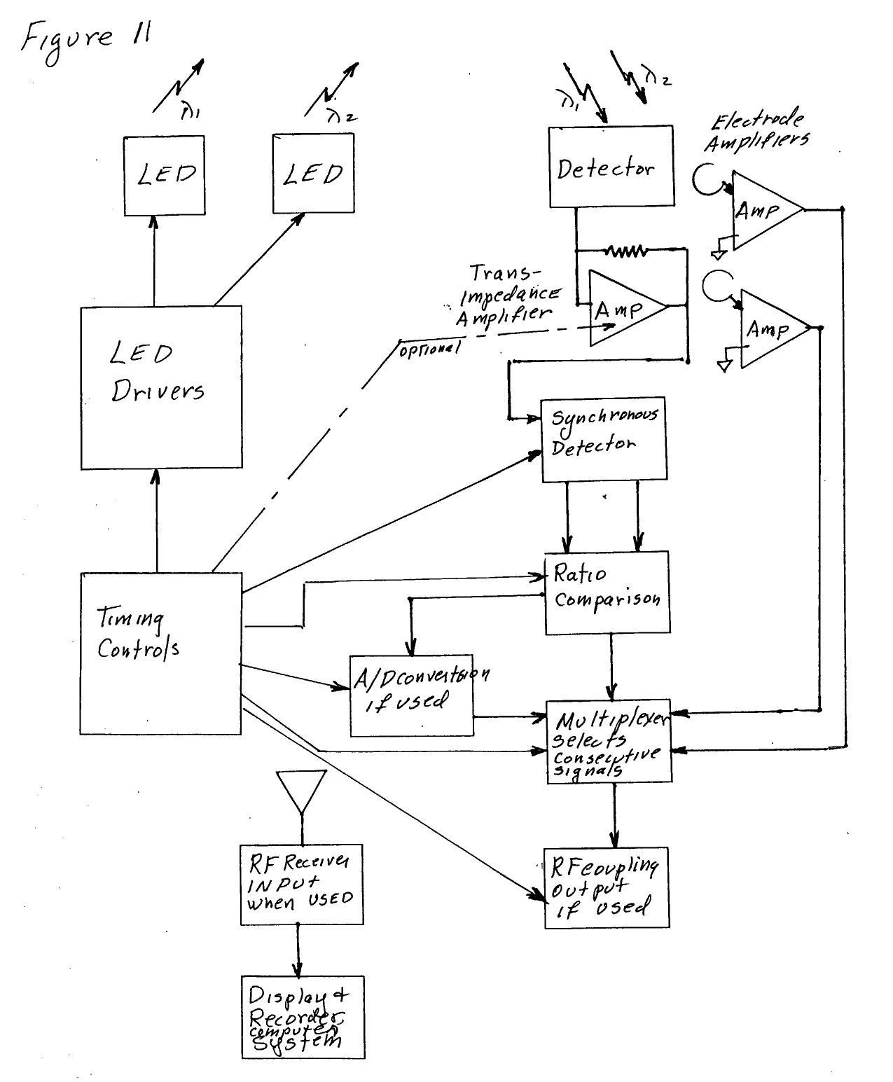

Various electronic circuits are possible to implement the acquisition of data from the sensor housing. FIG. 11 shows a typical functional circuit diagram to achieve the desired objective. Under timing control the LED drivers actuate the emission sources consecutively, which radiation, after passing through fetal tissue is detected and amplified. The detected outputs effectively constitute two or more voltages representative of the transmission wavelengths, which are ratioed to provide a normalized comparison for oxygen in the blood. Other parameter like glucose, albumen, etc. would have wavelength emissions related thereto. It should be noted that emitting an array of different wavelengths and combinations thereof might provide useful measured information in addition to blood oxygenation, without awareness as to exactly what “parameter” is being measured. Computer warnings signals could be provided by collecting a database of related tissue transmissions and electrical signals and subsequent “data mining” that data for statistical correlation with intrapartum events. For the RF coupling case, those analogue detector signals can be digitized by an analogue to digital converter and then ratioed digitally. The resultant signal, bearing correspondence to blood oxygen or whatever, goes to a multiplexer, which intersperses signals from the differential electrode amplifiers for EEG type information and common-mode to ground for EKG type signals if used. The resultant encoded sequence of signals modulates the radio frequency output radiation, intermittently under timing control, depending upon the desired information rate. The RF receiver remote to the housing picks up the signal and feeds it to a computer for analysis, discrimination, recording, comparison and display. FIG. 11 represents an example block diagram, which except for the LED's can be one ASIC chip. Numerous other circuitry arrangements are feasible to achieve a similar function. The above detailed description of various embodiments of the invention contains many specifics for purposes of illustration and enablement. Nevertheless, as the wide variation in embodiments demonstrates, this invention should not be determined by the specifics in these embodiments but by the following claims and their legal equivalents.

What is claimed is:

1) A means for monitoring fetal Oxygen status with a sensor;
A hollow-metal tissue-needle protruding from the sensor; said tissue-needle with two or more optical fibers within is disposed to penetrate the fetal epidermis.

2) A means as in claim 1 wherein one optical fiber is arranged to emit controlled optical radiation into the fetal tissue;
the other fiber is arranged to transfer a portion of said radiation transmitted through the fetal tissue to an opto-electronic detector;
said detector generating electrical signals commensurate with the radiation received.

3) An apparatus as in claim 1 wherein the outer metal-tubing of the tissue needle serves as electrode for an electrical signal associated with fetal status evaluation.

4) A method as in claim 1 wherein an acoustical sensor in proximity to the tissue needle converts prevailing acoustical signals into commensurate electrical signals.

5) A method as in claim 1 wherein one of the optical fibers receives controlled wavelength radiation from 2 or more LED die.

6) A means as in claim 2 wherein the detector is in the sensor;
response signals from the detector are disposed to be amplified within the sensor and modulate a radio frequency transmitter within the sensor.

7) A means as in claim 1 where the detector-generated signals are amplified and analyzed by computer algorithm for fetal vital signs such as blood oxygenation level.

8) An apparatus as in claim 1 where the optical fibers emerge through the birth canal.

9) A method for establishing fetal status where electrical, optical, and prevailing environmental conditions are measured and analyzed in relation to a data base derived from previous births;
said electrical and optical signals acquired from a fetal sensor attached to the fetus.

10) The method of claim 9 wherein the signals and data relate to one or more of the following:
date, time, place, fetal monitor employed, blood oxygen, glucose, albumen, hormones, enzymes, proteins, tissue electrical characteristics, EEG, heartbeat, images, acoustic sound, scattered light, temperature, vibration, airborne gas, atmospheric pressure, relative humidity, background magnetic, electromagnetic, ultrasound, electro kinetic signals, object accelerations, mother's age, weight, and ethnicity;
said measured data disposed to inter-compare with data from previous births.

11) A method as in claim 9 wherein the Internet is utilized for data exchange.

12) A method for monitoring fetal status parameters like heart rate and blood oxygenation comprised of;
one or more partially-metalized transparent-plastic tissue needle light pipes; wherein one or more light pipe is disposed to penetrate the epidermis of the fetus.

13) A method as in claim 12 wherein the light pipe connects to receiving electronics via one or more fiber optic cable that emerges from the womb through the birth canal.

14) A method as in claim 12 wherein metallization on a light pipe electrically connects to a signal lead that exits the birth canal.

15) A method as in claim 12 wherein metallization on a light pipe serves as an electrical signal conductor.

16) A method as in claim 10 wherein one or more amplifier is embedded within the housing of the fetal sensor.

17) A method as in claim 12 wherein an electrical signal from a metalized light pipe is analyzed by algorithm to evaluate status of the fetus.

18. A method as in claim 12 wherein one light pipe receives controlled optical radiation from 2 or more LED die;

19) A method as in claim 12 wherein one light pipe is disposed to transmit optical radiation from fetal tissue to an optoelectronic detector.

20) An apparatus as in claim 19 where the optoelectronic detector is within the fetal sensor.

21) An apparatus as in claim 19 where optical signals from the detector are amplified;
discriminant analysis type procedures to establish fetus status being brought to bear on said amplified signals.

22) A device for monitoring pulse oxygenation and fetal heart rate comprised of one or more fiber optic type light guide each surrounded by a hollow-metal tissue-needle disposed to penetrate fetal tissue.

23) The method of claim 22 wherein the light pipes have barbs to increase their adherence to the fetus when impressed upon its epidermis.

24) A method as in claim 22 wherein optical radiation from one light guide is disposed to impinge upon an optoelectronic detector.

25) An apparatus as in claim 22 wherein one light guide within the hollow-metal tissue-needle is an optical fiber that exits the birth canal.

26) An apparatus as in claim 22 where one light guide is disposed to emit optical radiation into fetal tissue;
a second light pipe is disposed to detect a portion of said radiation from the fetal tissue.

27) An apparatus as in claim 22 wherein one or more hollow-metal tissue-needle electrically connects to an amplifier input.

28) An apparatus as in claim 22 wherein one or more hollow-metal tissue-needle electrically connects to a lead wire exiting the birth canal.

29) An apparatus as in claim 22 wherein an optoelectronic detector is embedded within a sensor housing.

30) An apparatus as in claim 22 wherein two or more LED's emit controlled optical radiation into one or more fiber optic type light guide;

31) The method of claim 1 wherein a miniature C-MOS camera is disposed to view in the direction of the fetus enabling a display of the camera's field of view.

32) The method of claim 9 wherein a miniature C-MOS camera is disposed to view in the direction of the fetus enabling a display of the camera's field of view.

33) The method of claim 22 wherein a miniature C-MOS camera is disposed to view in the direction of the fetus enabling a display of the camera's field of view.

34) A method for fabricating a sensor used to ascertain optical and electrical properties of an object, wherein;
conductive metallization on a single partially-metalized transparent-plastic part is rigidly embedded within said sensor's housing;
said plastic part is then mechanically severed into two or more separate light pipes each respectively possessing a surrounding metallized conductor;
the distal end of said light pipes and electrodes being disposed to contact the test object.

35) The method of claim 34 wherein the sensor sends data to a receiver through a radio frequency link.

36) The method of claim 34 wherein the test object is a fetus.

37) The method of claim 34 wherein the mechanically severed light pipes with metallization are disposed to penetrate the surface of the test object.

38) The method of claim 37 wherein the severed light pipes penetrating the surface of the test object have barbs to increase adherence of the sensor to the object.

39) An apparatus to non-invasively monitor fetal status comprised of a bandage type sensor housing with two or more peripheral flaps;
said flaps disposed with adhesive to help bond the sensor to the fetus.

40) The apparatus of claim 39 wherein two or more LED die emit controlled wavelength radiation into the fetus through the front contact surface of the sensor;
an embedded optoelectronic sensor facing the front contact surface is disposed to detect a retro-reflected portion of said radiation.

41) The apparatus of claim 39 wherein battery power, control circuitry and an RF transmitter are disposed within the housing;
said circuitry enabling the processing of electrical signals to establish fetal status information at the receiving electronics.

42) The apparatus of claim 39 wherein Velcro-like fine wire needles pointing toward the fetus are embedded within the adhesive on the flaps of the sensor;
said wire needles disposed to increase adhesion to the fetus.

43) The apparatus of claim 39 wherein metallization on the front contact surface of the flaps serve as independent contacting electrodes to monitor heartbeat signals and brain wave type electrical signals of the fetus.

44) A method for inserting a bandage type sensor onto a fetus wherein the sensor is inserted into the womb with the flaps folded back parallel to the length of the insertion tool;
after contact of the sensor with the fetus, spring-like-extensions holding the flaps parallel to the insertion tool enable release of the flaps;
the outer concentric portion of the insertion tool is disposed to subsequently allow its distal end to pressure the flap's adhesive into improved contact with the fetus.

45) A method of fetal oxygen monitoring wherein two or more optical light guides pierce the fetal epidermis.

46) The method of claim 45 wherein one or more electrode is disposed to surround a portion of optical light guide;

47) A method for monitoring blood oxygenation utilizing hinged flaps with adhesive to hold the sensor onto the test subject.

48) A method of inserting a bandage type fetal sensor into the birth canal using an insertion tool;
wherein the Band-Aid type hinged flap extensions with contact-surface-adhesive can be subsequently pressed to the fetus.

49) A method of attaching a fetal-monitor sensor;
wherein one or more appendage tissue-needle on the sensor is constructed with sufficient flex that it can be spring loaded prior to fetal attachment;
spring pressure of said tissue-needle being directed toward entering fetal tissue partially transverse to the direction of sensor approach to the fetus upon contact with the fetus;
said transverse penetration of fetal tissue employed to hold the sensor onto the fetus.

50) A method for fabricating a sharp point on a hollow metal tissue needle filled with clear solid dielectric;
said method consisting of slicing said tissue-needle at a steep oblique-wedge angle relative to its axis to obtain a keen pointed edge.

51) A method of applying optical radiation to a fetus from one or more LED die within a sensor housing;
wherein the sensor adheres to the fetus via a tissue-needle.

52 A method of fetal monitoring and analysis utilizing the procedures indicated in U.S. Pat. No. 6,122,042.

53) A method of attaching a sensor to a fetus using one or more plastic tubes through the birth canal;
wherein at least one of said tubes serves as a light pipe illuminating the distal end.
